(19) United States
(12) Patent Application Publication
(10) Pub. No.: US 2020/0069868 A1
(43) Pub. Date: Mar. 5, 2020

(54) INJECTOR

(71) Applicant: MEDTRON AG, D-66128 SAARBRÜCKEN (DE)

(72) Inventor: Florian Bilz, Nalbach (DE)

(73) Assignee: MEDTRON AG, D-66128 SAARBRÜCKEN (DE)

(21) Appl. No.: 16/494,140

(22) PCT Filed: Mar. 12, 2018
(86) PCT No.: PCT/EP2018/056047

§ 371 Date: Sep. 13, 2019

(30) Foreign Application Priority Data
Mar. 15, 2017 (DE) 10 2017 105 481.2

(51) Int. Cl. A61M 5/145

(52) U.S. Cl. CPC A61M 5/1452; A61M 2005/14506

(57) ABSTRACT

An injector for injecting a liquid from a syringe with a movable syringe plunger. The injector includes a syringe receptacle for insertion of the syringe and, associated with the syringe receptacle, a drive unit for the syringe plunger. The drive unit is embodied as a spindle drive unit having a threaded spindle that can be driven around its longitudinal axis by a motor and having a spindle nut that is positioned on the threaded spindle, wherein the spindle nut is embodied as an unlocking slider with a through bore embodied as an oblong hole for the passage of the threaded spindle, whose maximum diameter extends perpendicular to the longitudinal axis of the threaded spindle and only in an end region of the maximum diameter, has a partial thread for the engagement in the threaded spindle, and the unlocking slider can be moved perpendicular to the longitudinal axis of the threaded spindle between an engagement position in which the partial thread engages with the threaded spindle and a release position in which the partial thread does not engage with the threaded spindle.

The invention relates to an injector for injecting a liquid from a syringe with a movable syringe plunger; the injector comprises a syringe receptacle for insertion of the syringe and, associated with the syringe receptacle, a drive unit for the syringe plunger; and the drive unit is embodied as a spindle drive unit having a threaded spindle that can be driven around its longitudinal axis by a motor and having a spindle nut that is positioned on the threaded spindle.

Injectors of this kind are known and are used, for example, for remote-controlled administration of contrast medium and other liquids as part of a computer tomography or magnetic resonance imaging, in relation to which reference is made, for example, to DE 197 14 711 A1.

DE 694 16 686 T2 has disclosed an injector with a drive unit embodied as a spindle drive unit for actuating a syringe that is inserted into the syringe receptacle and has a movable syringe plunger.

Such injectors must meet high standards with regard to simple handling so that they can be quickly and easily prepared for an injection that is to be carried out and can be quickly readied for use again between successive patient examinations. The insertion of the syringe, which is filled with the liquid to be injected, should occur in the easiest possible way and it should also be possible to remove the empty syringe quickly. In this connection, however, the problem often arises that particularly with drive units embodied as spindle drive units, because of the selected small thread pitch of the threaded spindle, after a previously inserted syringe has been emptied, the return movement of such a drive unit takes a very long time, which seems in need of improvement.

The object of the invention, therefore, is to propose an injector of the type mentioned at the beginning, which can be handled with particular ease and speed and, despite a simple mechanical design, overcomes the disadvantages of the prior art.

To attain the stated object, the invention proposes the embodiment of an injector with the features of claim 1. Advantageous embodiments and modifications of the invention are the subject of the dependent claims.

The invention proposes the embodiment of the spindle nut as an unlocking slider with a through bore embodied as an oblong hole for the passage of the threaded spindle, whose maximum bore diameter extends perpendicular to the longitudinal axis of the threaded spindle and only in an end region of the maximum diameter, is provided with a partial thread for engagement in the threaded spindle, and the unlocking slider can be moved perpendicular to the longitudinal axis of the threaded spindle between an engagement position in which the partial thread engages with the threaded spindle and a release position in which the partial thread does not engage with the threaded spindle.

According to the invention, it is therefore possible to simply switch the injector between two operating positions determined by the unlocking slider. In the first operating position in which the unlocking slider is in the engagement position, the partial thread engages in the threaded spindle and thus enables the function of the drive unit, as a spindle drive unit, to move the syringe that is inserted into the syringe receptacle. By switching into the second operating position in which the unlocking slider is in the release position, the partial thread no longer engages in the threaded spindle and the function of the spindle drive unit is disabled, which results in the fact that the unlocking slider and all of the other components connected to it can be freely moved, for example by hand, along the longitudinal axis of the threaded spindle. It is thus possible, for example after a syringe that is inserted into the syringe receptacle has been emptied, to manually return the drive unit very quickly and with an extremely simple operation.

According to one proposal of the invention, the unlocking slider is elastically prestressed in the direction of the engagement position so that it seeks to engage in the thread turns of the threaded spindle in the manner of a spindle drive unit. By means of a deliberate switching procedure, for example by an operator, this state can be temporarily canceled in opposition to the elastic prestressing, for example in order to return the drive unit by hand. The elastic prestressing of the unlocking slider can be produced, for example, by means of a spring assembly.

According to another proposal of the invention, the prestressing of the unlocking slider can be dimensioned so that when a predeterminable threshold is exceeded, the unlocking slider is moved out of the engagement position and into the release position in opposition to the elastic prestressing. For example, if during the advancing movement of the spindle drive unit with the unlocking slider in the engagement position, an increased pressure load on the drive unit of the injector occurs due to a mechanical problem or due to an interruption in the flow to the patient from the syringe that is inserted into the injector, then the partial thread of the unlocking slider is moved along the engaged thread flanks in the direction of the release position and the unlocking slider—due to the still-resent elastic prestressing—jumps over into the next subsequent thread turn of the threaded spindle so that an automatic overload protection is achieved. Furthermore, in a way that will be described in greater detail below, this temporary displacement into the release position can be detected with corresponding sensors in order to signal an overload situation to a control system and, for example, to trigger an automatic shut-off and/or an alarm.

As a partial thread in the sense of the invention, preferably a half-thread is provided in one of the two end regions of the maximum diameter inside the oblong hole in the unlocking slider.

According to one proposal of the invention, the threaded spindle is supported in the injector in a rotatable, stationary fashion and the unlocking slider is positioned so that it is able to move along the longitudinal axis of the threaded spindle and is connected to a thrust tube, which is slid part-way onto the threaded spindle and which, on the syringe receptacle, is associated with the syringe plunger. The thrust tube produces the connection to the syringe plunger inside the syringe that is inserted in the syringe receptacle and, when the threaded spindle 22 rotates and the unlocking slider is in the engagement position, is moved by it along the longitudinal axis of the threaded spindle in the manner of a spindle drive unit and presses on the syringe plunger so that ultimately, the liquid is injected from the syringe in the desired way.

According to one proposal of the invention, the unlocking slider and the thrust tube are accommodated in a sliding bridge, which is guided in sliding fashion along the longitudinal axis of the threaded spindle and which can be moved together with the accommodated unlocking slider along the longitudinal axis of the threaded spindle.

According to another proposal, the thrust tube is embodied with a switch cam at its end oriented toward the unlocking slider, by means of which the unlocking slider can be moved between the release position and the engagement position upon rotation of the thrust tube around the longitudinal axis of the threaded spindle. In this respect, the thrust tube has a double function and serves not only to transmit force to the syringe plunger, but also—when rotated around its longitudinal axis that coincides with the longitudinal axis of the threaded spindle—causes a rotation of the formed-on or mounted switch cam, as a result of which the unlocking slider, depending on the position of the switch cam, is moved into the engagement position or the release position. The thrust tube thus also performs the function of the actuating mechanism for the displacement of the unlocking slider out of the engagement position into the release position and vice versa.

According to another proposal of the invention, at its end oriented away from the unlocking slider, the thrust tube can be embodied with a lever for the rotation around the longitudinal axis of the threaded spindle, which simplifies the operation.

Finally, according to another proposal of the invention, a sensor for detecting the release position and the engagement position of the unlocking slider can be provided and by means of this sensor, a measurement signal can be produced, which correlates to the release position and/or the engagement position.

Such a measurement signal can, for example, be evaluated by a control unit of the injector according to the invention in order to prevent the drive unit from starting when the unlocking slider is in the release position, in order to also issue a corresponding alarm message when the unlocking slider is in the release position, and in order to trigger a shut-off of the drive unit and/or trigger an alarm when the unlocking slider, which is in the engagement position, jumps over into the subsequent thread turn in the above-described way due to an overload situation.

In the above-mentioned context, a sensor can, for example, be a microswitch or microbutton, which contacts the unlocking slider, for example by means of a contact pin. Such a contact pin can also simultaneously serve as a central guide pin for a coil spring that produces the elastic prestressing on the unlocking slider.

Other embodiments and details of the invention will be explained below based on the drawings, which show an exemplary embodiment. In the drawings:

FIG. 1 shows a perspective depiction of an injector according to the invention with the casings removed;

FIG. 2 shows a drive unit of the injector according to FIG. 1;

FIG. 3 shows a section through the drive unit according to FIG. 2 in the engagement position;

FIG. 4 shows a section through the drive unit according to FIG. 2 in the release position;

FIG. 5 shows a top view of a thrust rod of the drive unit according to FIG. 2;

FIG. 6 shows the end view of the thrust rod according to FIG. 5;

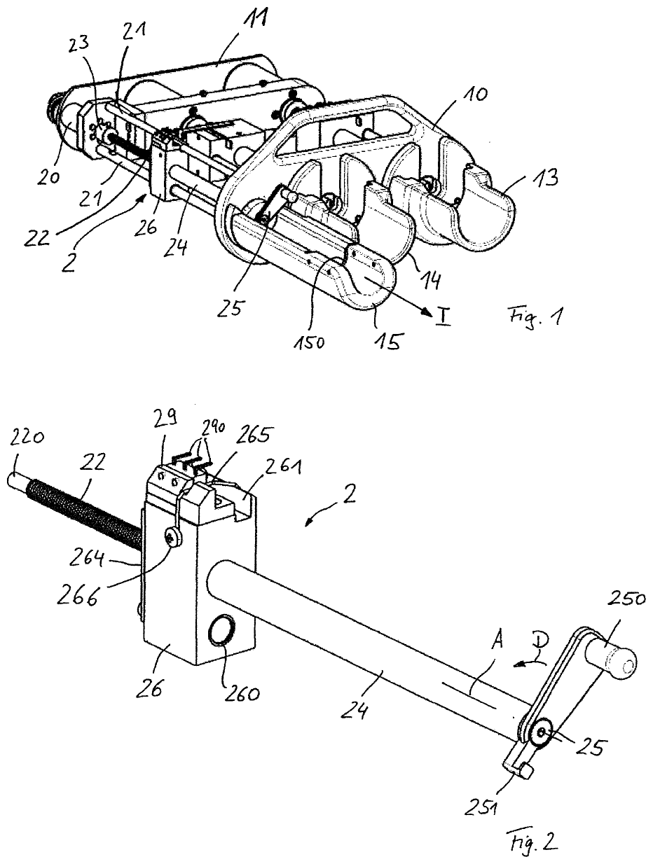

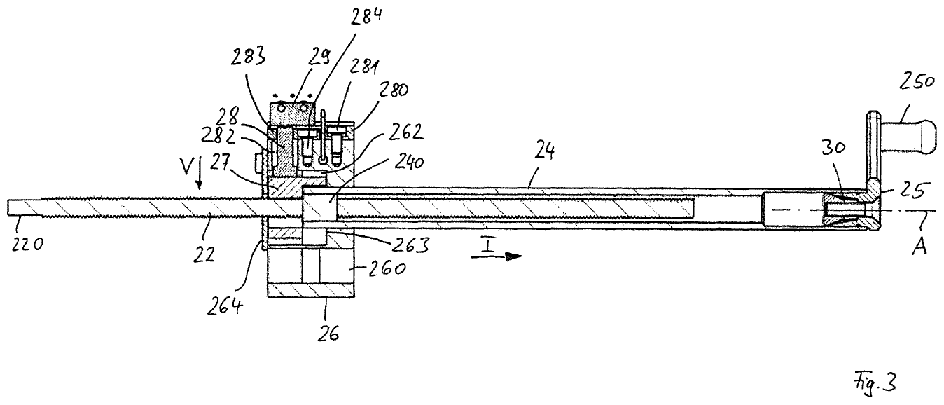

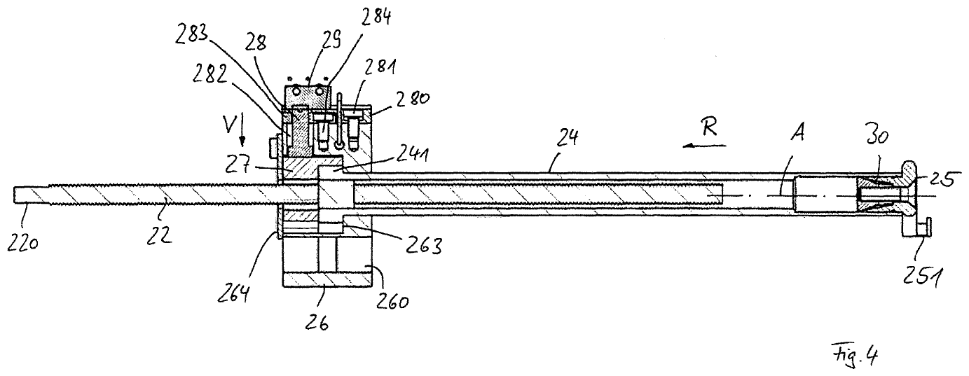

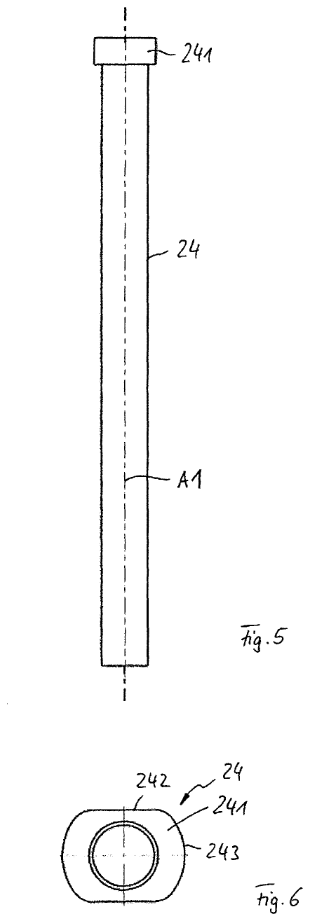

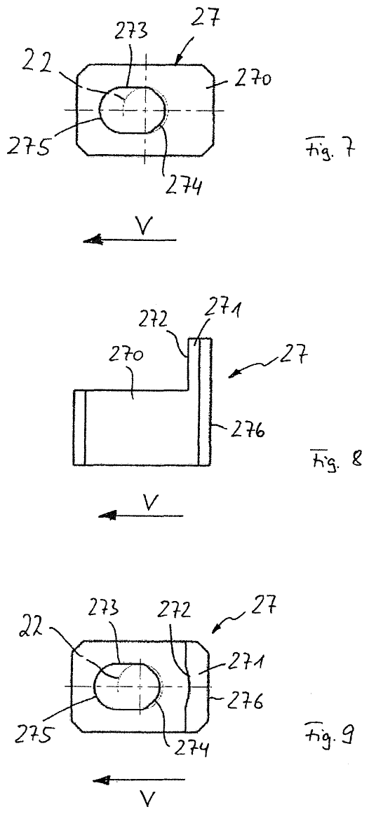

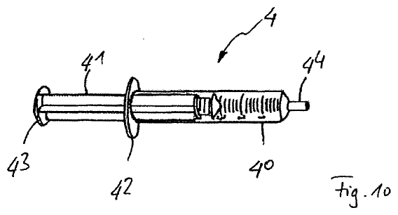

FIGS. 7-9 show various views of the unlocking slider according to the invention;

FIG. 10 shows a view of a syringe according to the prior art.

FIG. 1 shows a perspective depiction of an exemplary embodiment of a medical injector in which parts of the otherwise provided casing have been removed for better visibility. The injector shown in the exemplary embodiment according to FIG. 1 is used for injecting liquids from syringes, not shown here, for example as part of an MRI or CT scan in which a contrast medium and saline solution as a rinsing solution are usually administered.

For this purpose, the injector shown in FIG. 1 comprises a front wall 10 in front which there are two essentially identically embodied syringe receptacles 13, 14 and a third syringe receptacle 15, into which correspondingly configured syringes filled with the liquids to be injected (not shown) can be inserted and then, initiated by means of a control unit, injections can be performed in the way described below by drive units 2 positioned between the front wall 10 and a back wall 11.

The third syringe receptacle labeled with the reference numeral 15 in the exemplary embodiment shown is used, for example, to accommodate and inject a drug that is needed for the examination, e.g. adenosine for initiating a stress test of the patient being examined. It is understood, however, that the statements made in this application with regard to the further embodiment of the drive unit 2 of the syringe receptacle 15 can also apply equally to the other drive units 2 of the syringe receptacles 13, 14 and a thus-equipped injector can also have different numbers of syringe receptacles than the total of three syringe receptacles 13, 14, and 15 shown here. In the following, however, the design of the drive unit 2, which is associated with the syringe receptacle 15, will be explained in greater detail without being restricted to this syringe receptacle 15.

Conventional syringes 4 are used for accommodating and injecting the liquid to be administered as part of the examination to be performed; FIG. 10 shows an example of such an embodiment of a syringe 4.

In an intrinsically known way, the syringe 4 consists of a syringe cylinder 40 with a conical nozzle 44 to which a tube for connecting to the patient can be attached and at the end opposite from the nozzle 44, a syringe plunger 41 that is inserted into the syringe cylinder 40 in a movable way. The movement of the syringe plunger 41 is produced by pushing on the pushing surface 43 at the end; the syringe cylinder 40 has a collar 42 formed onto it to produce a corresponding counter-pressure.

To match this configuration of a syringe 4, the syringe receptacle 15 of the injector according to the depiction in FIG. 1 is embodied so that the syringe plunger 40 can be inserted into the syringe receptacle 15 that is embodied in the form of a semicylindrical shell, with the collar 42 engaging in a form-fitting way in a groove 150 of the syringe receptacle 15. The actuation of a drive unit in the arrow direction I, which is explained in greater detail below, pushes a contact surface 25 of the drive unit 2 in the desired way against the pushing surface 43 of the syringe plunger 41 and produces the injection of the liquid stored inside the syringe cylinder 40 through the nozzle 44 in the direction toward the patient.

The drive unit 2 of the injector, which produces the forward displacement of the contact surface 25 in direction I, is also shown in greater detail in FIGS. 2 through 4.

The drive unit 2 comprises a threaded spindle 22, whose longitudinal axis A extends in the arrow direction I and is embodied with an external thread with a preferably small thread pitch. At its end oriented toward the back wall 11 of the injector, the threaded spindle 22 is provided with a spindle pin 220, which is accommodated in a rotationally coupled fashion in a bushing 23 and can be driven via a transmission 20 by an external drive motor that is not shown here in order to be able to rotate the threaded spindle 22 around its longitudinal axis A in motor-driven fashion.

The drive unit 2 also comprises a sliding bridge 26 that can be moved along cylindrical guide rods 21 and that can be made, for example, of an injection-molded plastic and has a through bore 260 as well as a recess 261 at the top to permit the guide rods 21 to pass through and provide guidance. The guide rods 21 extend parallel to the longitudinal axis A and thus define a mobility of the sliding bridge 26 in the arrow direction I and opposite from it.

The sliding bridge 26, as is visible in greater detail in FIGS. 3 and 4, has a receiving space 262, which accommodates an unlocking slider 27 that can be seen in greater detail in FIGS. 7 to 9. The size of the receiving space 262 is dimensioned so that the unlocking slider 27 can be moved perpendicular to the longitudinal axis A of the threaded spindle 22, i.e. in the exemplary embodiment shown, in the arrow direction V and in the opposite direction at right angles to the longitudinal axis A. The receiving space 262 with the unlocking slider 27 accommodated therein is closed by means of a covering plate 264 that is fastened to the sliding bridge 26 with screws.

The unlocking slider 27, which can in turn likewise be made of a suitable injection-molded plastic, has an approximately block-shaped base body 270 with a protruding cantilever arm 271 formed onto it, which extends parallel to a through bore 273 through which the threaded spindle 22 is guided in the installed state according to FIGS. 3 and 4.

The through bore 273 of the unlocking slider 27 is embodied as an oblong hole with a larger diameter and a smaller diameter, with the smaller diameter being selected to be slightly larger than the outer diameter of the threaded spindle 22 and the larger diameter being enlarged relative to it enough to yield a sufficient movement path in the arrow direction V and in the opposite direction when the threaded spindle 22 is guided through the through bore 273. In the exemplary embodiment shown, the ratio of the smaller diameter of the through bore 273 to the larger diameter of the through bore 273 is 1:1.5, where the larger diameter extends in the arrow direction V and thus perpendicular to the longitudinal axis A of the threaded spindle 22.

In an end region 274 of the maximum diameter of the through bore 273, a partial thread that is matched to the external thread of the threaded spindle 22, e.g. a half-thread, is provided in the form of an internal thread section, which allows the unlocking slider 27 as a spindle nut, to engage in the external thread of the threaded spindle 22 and when the threaded spindle 22 rotates around its longitudinal axis A, to be moved along this longitudinal axis A.

In the opposite end region 275 of the maximum diameter of the through bore 273, however, there is no corresponding thread; in this region, the wall of the through bore 273 is instead embodied as smooth.

It is thus possible for the unlocking slider 27, because of its ability to move in the arrow direction V and in the opposite direction inside the receiving space 262 of the sliding bridge 26, to assume two different positions, which correspond to two different switch states and are which are contrasted in FIGS. 3 and 4.

In FIG. 3, the unlocking slider 27 is shown in an engagement position in which it is moved downward in the arrow direction V until the half-thread 274 of the through bore 273 engages in the external thread of the threaded spindle 22. If the threaded spindle 22 is motor-driven in the above-described way to rotate around its longitudinal axis A in the corresponding rotation direction, then in this engagement position, the unlocking slider 27 travels on the threaded spindle 22 like a spindle nut along the longitudinal axis A, for example in the direction of the arrow I.

It is also clear that on the side of the threaded spindle 22 oriented away from the spindle pin 220, a thrust tube 24 that concentrically encloses the threaded spindle 22 is slid on, which is likewise accommodated in the sliding bridge 26 with its one end resting laterally against the unlocking slider 27, and in the region of this end, is guided on the threaded spindle 22 in sliding fashion by means of an inserted slide bushing 240.

When the unlocking slider 27 that functions as a spindle nut moves in the arrow direction I due to a rotation of the threaded spindle 22, this movement is correspondingly transmitted to the sliding bridge 26 and the thrust tube 24 whose end oriented away from the sliding bridge 26, by means of a clamping cone 30, supports the contact surface 25 for engaging the syringe plunger 43.

It is therefore clear that when the unlocking slider 27 is in the engagement position according to FIG. 3, by means of a motor rotation of the threaded spindle 22, a forward displacement of the thrust tube 24 and the contact surface 25 embodied at the front end thereof along the longitudinal axis A of the threaded spindle 22 in the arrow direction I causes the liquid to be injected from the syringe 4 that is inserted into the syringe holder 15.

If the unlocking slider 27 is lifted in the direction opposite from the arrow direction V into its release position inside the receiving space 262 of the sliding bridge 26 as shown in FIG. 4, then the half-thread 274 of the through bore 273 correspondingly no longer engages with the external thread of the threaded spindle 22. As a result, the function of the unlocking slider 27 as a spindle nut on the threaded spindle 22 is canceled and, for example after completion of an injection procedure, the unit composed of the unlocking slider 27, the sliding bridge 26, and the thrust rod 24 can be quickly moved back into this release position in the arrow direction R, for example by hand, in order to remove an empty syringe and to prepare the syringe receptacle 15 for the insertion of a new, full syringe 4 with a syringe plunger 41 protruding a corresponding distance from the syringe cylinder 40. It is also possible in the reverse direction, after the insertion of an only partially filled—for example half-filled—syringe 4, to quickly move the thrust rod 24 and the sliding bridge 26 with the unlocking slider 27 connected to it in the arrow direction I and place it against the syringe plunger 41 that is protruding part-way out from the syringe cylinder 40. This simplifies the handling of the drive unit 2 enormously.

The placement of the unlocking slider 27 in the engagement position according to FIG. 3 or in the release position according to FIG. 4 and the movement required for the change in position are produced in a particularly simple way by means of the thrust tube 24. For this purpose, the thrust tube 24, as can be seen in the depictions in FIGS. 5 and 6, has an enlarged region embodied in the form of a switching cam 241 at its end that is accommodated in the sliding bridge 26, in which two diametrically opposed cylindrical sections 243 with a larger diameter are connected by two diametrically opposed edges 242 offset from them by 90°, which that have the smaller diameter.

The enlarged region embodied in the form of a switching cam 241 on the one hand produces the captive securing of one end of the thrust tube 24 in the sliding bridge 26, which for this purpose has a step-shaped bore 263 that adjoins the receiving space 262 and that is engaged from behind by the switching cam 241 that comes to rest inside the receiving space 262.

On the other hand, however, the switching cam 241 also produces the movement and positioning of the unlocking slider 27 inside the receiving space 262. For this purpose, in the region of the underside of its protruding cantilever arm 271, the unlocking slider 27 has a sliding surface 272 that is adapted to the switching cam 241 so that through the rotation of the thrust tube 24 together with the switching cam 241 accommodated in the receiving space 262 around the axis A, the unlocking slider 27 is moved in the arrow direction V and in the opposite direction, between its engagement position according to FIG. 3 and its release position according to FIG. 4. If the sliding surface 272 is resting against a straight edge 242 of the switching cam 241, this corresponds to the depiction according to FIG. 3 and the unlocking slider 27 is in the engagement position. But if the thrust tube 24 is rotated by 90° around its longitudinal axis that coincides with the longitudinal axis A of the threaded spindle 22, then the cylindrical section 243 of the switching cam 241 with the correspondingly larger radius rests against the sliding surface 272 of the unlocking slider 27 and lifts it in the direction opposite from arrow V out of the engagement position shown in FIG. 3 into the release position shown in FIG. 4 so that the thread engagement with the threaded spindle 22 is canceled. When the thrust tube 24 is rotated back again by 90°, the unlocking slider 27 once again arrives into its engagement position according to FIG. 3.

The thrust tube 24 thus performs a double function, namely on the one hand, that of transmitting force from the unlocking slider 27 serving as a spindle nut to the syringe plunger 41 and on the other hand, that of the switching mechanism for the unlocking slider 27 and its movement between the release position and the engagement position.

In order to facilitate the above-explained rotation of the thrust tube 24 by 90° in order to move the unlocking slider 27, a lever 250 for rotating the thrust tube 24 around its longitudinal axis and thus also around the longitudinal axis A of the threaded spindle 22 is formed onto the front end with the contact surface 25.

Formed onto the side diametrically opposite from the lever 250 relative to the longitudinal axis, there is a hook 251, which engages around the pushing surface 43 of a syringe 4 that is inserted into the syringe receptacle 15.

In order for the movement of the unlocking slider 27 between the engagement position in FIG. 3 and the release position in FIG. 4 to be precisely tracked in accordance with the position of the switching cam 241, above the unlocking slider 27 in the sliding bridge 26, a contact pin 28 is provided, which rests against on a pushing surface 276 of the unlocking slider 27 and is elastically prestressed against the unlocking slider 27 in the arrow direction V by a coil spring, not shown, that is positioned in a spring chamber 282 surrounding the contact pin 28. An appropriate counter-support is provided in the form of a spring cover 283 that is mounted to the top of the sliding bridge 26 by means of a screw 284. Because of the elastically prestressed contact pin 28, the unlocking slider 27 is also elastically prestressed against the threaded spindle 22 in the arrow direction V and thus into the engagement position shown in FIG. 3 and, through the contact of the cylindrical cam surface 243 of the switching cam 241 against the sliding surface 272, is lifted into the release position shown in FIG. 4 in opposition to the spring force, but seeks to return to the engagement position according to FIG. 3.

In addition to the above-explained switching of the unlocking slider 27 between the engagement position and the release position that is produced by user intervention, the unlocking slider 27, which is elastically prestressed in this way, also offers an automatically acting overload protection of the drive unit 2. If, during the injection or during the extending of the thrust tube 24 in the arrow direction I that produces the injection, a malfunction occurs, for example due to the syringe plunger 41 becoming jammed in the syringe cylinder 40 or due to a flow interruption in the tube section between the nozzle 44 and the patient, then as the threaded rod 22 rotates further, the counter-pressure that builds up in the arrow direction R causes there to be a danger of an overload of the drive unit 2, with the consequence of a possible mechanical damage or an excessive pressure load for the patient.

If a limit force in the arrow direction R—which depends on the thread geometry between the threaded spindle 22 and the unlocking slider 27 and on the spring force of the coil spring acting on the contact pin 28—is exceeded, then this causes the thread flanks of the half-thread 274, which are in engagement with the threaded spindle 22, to move upward on the thread flanks of the threaded spindle 22. As a result, the unlocking slider 27 is temporarily lifted in opposition to the prestressing by the contact pin 28, in the direction opposite from the arrow direction V and toward the release position, and ends up jumping one thread turn in the arrow direction R until, through the action of the elastically prestressed contact pin 28, it moves back in the arrow direction V into the engagement position. This procedure repeats until the force falls below the limit force in the arrow direction R. This therefore prevents an overloading of the drive unit 2 and/or of the patient in a reliable and fully automatic way.

Above the contact pin 28, there is also a sensor 29, which monitors the contact pin 28 and is embodied in the form of a microswitch, whose connections 290 can be connected to signal lines of a control unit, not shown in detail here, for the injector. In the unloaded switch state of the sensor 29, the unlocking slider and the contact pin 28 that acts on it in a spring-elastic fashion are in the lower engagement position according to FIG. 3. Through a corresponding signal at the connections 290, the control unit can signal the engagement position and thus the operational readiness of the unlocking slider 27 and the entire drive unit 2, and an injection can be carried out accordingly.

But if, starting from the depiction in FIG. 3, the thrust tube 24 is rotated by 90° relative to the longitudinal axis and assumes the orientation according to FIG. 4, then by means of the cylindrical cam surface 243, the switching cam 241 pushes the unlocking slider 27 and the contact pin 28 upward in the direction opposite from the arrow direction V, as a result of which the sensor 29 positioned on the contact pin 28 is correspondingly actuated. By means of a corresponding different signal at the connections 290, the control unit can signal the release position and thus an interruption in the operation of the unlocking slider 27 and of the entire drive unit 2, and an injection can be correspondingly prevented.

In the same way, the sensor 29 also registers the above-explained overload-induced jumping of the unlocking slider 27 over individual thread turns of the threaded spindle 22 since this causes temporary assumption of the release position and the sensor 29 is thus triggered accordingly by means of the synchronously moved contact pin 28. Through a correspondingly emitted signal at the connections 290, the control unit can be informed of the overload situation during the ongoing injection procedure and it can trigger an immediate shut-off of the drive unit and/or trigger a corresponding alarm.

Since the vertical stroke—which the unlocking slider 27 and the contact pin 28 execute in the direction opposite from the arrow direction V in the switch between the engagement position and the release position—is usually larger than the button travel of a sensor 29 that is embodied for example as a microswitch, the sensor 29 is fastened together with a contact cover 280 in an elastically flexible fashion opposite from the arrow direction V on the sliding bridge 26 by means of the screw 281. This assembly is elastically prestressed in the arrow direction V, for example by means of a rubber cord 265 that is clamped by means of the contact cover 280 and fastened to the sliding bridge 26 by means of screws 266.

The above-explained drive unit 2 of an injector thus enables a particularly simple use that is protected from operating errors. If the lever 250 on the thrust tube 24 is in the orientation shown in FIGS. 1, 2, and 3, then a pre-filled syringe 4 can be inserted into the syringe receptacle 15 and subsequently emptied in the direction of the patient by means of a motor-driven advancing of the thrust tube 24 in the arrow direction I. Through rotation of the lever 250 in the arrow direction D according to FIG. 2 into an orientation that is offset by 90°, which is shown in FIG. 4, the unlocking slider 27 is moved out of the engagement position into the release position and is uncoupled from the action of a spindle nut so that the entire unit composed of the thrust tube 24, the sliding bridge 26, and the unlocking slider 27 accommodated therein can be slid by hand back in the arrow direction R into the starting position according to FIG. 1. It is therefore not necessary for the thrust tube 24 to be conveyed back into the starting position in motor-driven fashion through the return movement of the threaded spindle 22, which, due to the predominantly small thread pitch, would usually have to be accompanied by high speeds of the threaded spindle 22 and/or take an undesirably long time.

The drive unit 2 also offers a reliable, automatic, and purely mechanical overload protection, which is permanently active without an external control intervention and independent of the energy supply so that the greatest possible patient safety is achieved.

The injector according to the invention can be predominantly made of non-magnetic materials so that it can also be harmlessly used in proximity to powerful magnetic fields such as those of an MRI machine.

1. An injector for injecting a liquid from a syringe (4) with a movable syringe plunger (41); the injector (1) including a syringe receptacle (15) for insertion of the syringe and, associated with the syringe receptacle (15) a drive unit (2) for the syringe plunger (41); and the drive unit (2) embodied as a spindle drive unit having a threaded spindle (22) that can be driven around a longitudinal axis (A) by a motor and having a spindle nut that is positioned on the threaded spindle (22), the injector comprising: the spindle nut embodied as an unlocking slider (27) with a through bore (273) embodied as an oblong hole for passage of the threaded spindle (22), whose maximum diameter extends perpendicular to the longitudinal axis (A) of the threaded spindle (22) and only in an end region of the maximum diameter having a partial thread (274) for the engagement in the threaded spindle (22), and the unlocking slider (27) movable perpendicular to the longitudinal axis (A) of the threaded spindle (22) between an engagement position in which the partial thread (274) engages with the threaded spindle (22) and a release position in which the partial thread (274) does not engage with the threaded spindle (22).

2. The injector according to claim 1, wherein the unlocking slider (27) is elastically prestressed in a direction toward the engagement position.

3. The injector according to claim 2, wherein a prestressing of the unlocking slider (27) is dimensioned so that when a predeterminable threshold is exceeded, the unlocking slider (27) is moved out of the engagement position and into the release position in opposition to the elastic prestressing.

4. The injector according to claim 2, wherein the threaded spindle (22) is supported in the injector in a rotatable stationary fashion and the unlocking slider (27) is positioned so that it is movable along the longitudinal axis (A) of the threaded spindle (22) and is connected to a thrust tube (24) slid part-way onto the threaded spindle (22) and which on the syringe receptacle is associated with the syringe plunger (41).

5. The injector according to claim 3, wherein the unlocking slider (27) and the thrust tube (24) are accommodated in a sliding bridge (26) which is guided in sliding fashion along the longitudinal axis (A) of the threaded spindle (22).

6. The injector according to claim 4, wherein the thrust tube (24) has a switch cam at an end oriented toward the unlocking slider (27), by which the unlocking slider (27) is movable between the release position and the engagement position upon rotation of the thrust tube (24) around the longitudinal axis (A) of the threaded spindle (22).

7. The injector according to claim 5, wherein at an end oriented away from the unlocking slider (27), the thrust tube (24) has a lever (250) for the rotation around the longitudinal axis (A) of the threaded spindle (27).

8. The injector according to claim 7, wherein a sensor (29) for detecting the release position and the engagement position of the unlocking slider (27) is provided and by the sensor (29) it is possible to produce a measurement signal that correlates to the release position and the engagement position.

9. The injector according to claim 1, wherein the threaded spindle (22) is supported in the injector in a rotatable stationary fashion and the unlocking slider (27) is positioned so that it is movable along the longitudinal axis (A) of the threaded spindle (22) and is connected to a thrust tube (24) slid part-way onto the threaded spindle (22) and which on the syringe receptacle is associated with the syringe plunger (41).

10. The injector according to claim 9, wherein the unlocking slider (27) and the thrust tube (24) are accommodated in a sliding bridge (26) which is guided in sliding fashion along the longitudinal axis (A) of the threaded spindle (22).

11. The injector according to claim 3, wherein the thrust tube (24) has a switch cam at an end oriented toward the unlocking slider (27), by which the unlocking slider (27) is movable between the release position and the engagement position upon rotation of the thrust tube (24) around the longitudinal axis (A) of the threaded spindle (22).

12. The injector according to claim 11, wherein at an end oriented away from the unlocking slider (27), the thrust tube (24) has a lever (250) for the rotation around the longitudinal axis (A) of the threaded spindle (27).

13. The injector according to claim 1, wherein a sensor (29) for detecting the release position and the engagement position of the unlocking slider (27) is provided and by the sensor (29) it is possible to produce a measurement signal that correlates to the release position and the engagement position.
